(19) United States
(12) Patent Application Publication
(10) Pub. No.: US 2020/0395118 A1
(43) Pub. Date: Dec. 17, 2020

(54) ELECTRONIC IDENTIFICATION TAGGING SYSTEMS, METHODS, APPLICATORS, AND TAPES FOR TRACKING AND MANAGING MEDICAL EQUIPMENT AND OTHER OBJECTS

(71) Applicants: Westin Hill, Charlestown, MA (US); Duke University, Durham, NC (US)

(72) Inventors: Patrick Codd, Durham, NC (US); Westin Hill, Charlestown, MA (US); Ian Hill, Durham, NC (US); Weston Ross, Durham, NC (US); Matthew Tucker, Durham, NC (US)

(21) Appl. No.: 17/004,062

(22) Filed: Aug. 27, 2020

Related U.S. Application Data
(63) Continuation of application No. PCT/US19/21324, filed on Mar. 8, 2019.
(60) Provisional application No. 62/640,107, filed on Mar. 8, 2018.

(51) Int. Cl. G16H 40/20; G06K 7/10

(52) U.S. Cl. CPC G16H 40/20; G06K 7/10366

(57) ABSTRACT

Electronic identification tagging systems, methods, applicators, and tapes for tracking and managing medical equipment and other objects are disclosed. According to an aspect, a system includes electronic identification tag readers distributed within predetermined areas of an environment. The system also includes electronic identification tags attached to respective medical equipment within the environment. Further, the system includes a computing device comprising an object use analyzer configured to receive, from the electronic identification tag readers, information indicating presence of the electronic identification tags within the predetermined areas. The object use analyzer also analyzes usage of the medical equipment within the environment based on the received information. Further, the object use analyzer manages one of medical equipment supply or usage of the medical equipment during a medical procedure based on the analyzed usage of the medical equipment.

### CROSS REFERENCE TO RELATED APPLICATIONS

This continuation patent application claims priority to PCT Patent Application No. PCT/US19/21324, filed Mar. 8, 2019, and titled ELECTRONIC IDENTIFICATION TAGGING SYSTEMS, METHODS, APPLICATORS, AND TAPES FOR TRACKING AND MANAGING MEDICAL EQUIPMENT AND OTHER OBJECTS, which claims priority to U.S. patent application Ser. No. 62/640,107, filed Mar. 8, 2018, and titled DEVICES, SYSTEMS AND METHODS FOR INSTRUMENT TRACKING, the contents of which are incorporated herein by reference in their entireties.

### TECHNICAL FIELD

The presently disclosed subject matter relates generally to healthcare. Particularly, the presently disclosed subject matter relates to electronic identification tagging systems, methods, applicators, and tapes for tracking and managing medical equipment and other objects.

### BACKGROUND

Operating rooms (ORs) generate both the largest revenue and incur the greatest cost for the hospital. Their efficiency is essential to providing a high level of care at an affordable cost to the patient. Surgical instrument management and management of other medical equipment has been recognized as an area in need of improvement. 31% of a hospital's expense per case is attributed to supplies. Excessive instrumentation, manual instrument counts, and mismanagement can delay the operation, increase the workload of hospital staff, and introduce significant cost to the hospital and patient. These deficiencies are largely due to the lack of real time location transparency for surgical instruments. Less than 3% of hospitals have a tracking system, yet the United States Food and Drug Administration (FDA) requires that by Sep. 24, 2020, all hospitals in the United States must label each piece of equipment used in surgical operations with a unique device identifier. This mandate provides motivation to hospital management to implement tracking systems aimed at improving the efficiency of instrument management through the eradication of oversupply and missing instrumentation and other medical equipment.

In order to ensure successful operations while maintaining schedule, surgeons can sometimes request an excess of instruments in the OR. An estimated 78-87 percent of instruments in the OR go unused, introducing dramatic cost to the hospital in the form of cleaning and processing (estimated to be greater than $0.51 per instrument), delayed surgical operations, increased workload of nursing assistants, and unnecessary instrument wear. Concurrent with this drastic oversupply, it is estimated that approximately 1.6-5.9 percent of a surgeon's procedure time is spent waiting for an instrument that is not immediately available, which can be both frustrating and dangerous to the patient. Clearly, there is a balance to how many and which type of instrumentation should be supplied in the OR that optimizes the cost and the time efficiency of the operation. This balance has not yet been discovered as there is little data on which instruments are used.

Oversupply also contributes to the prevalence of retained surgical instruments and missing instrumentation. There are approximately 1500 instances of retained surgical instruments (RSI) in the United States every year. The Joint Commission estimates that the cost of additional medical care is over $166,000 and the medical liability cost is over $200,000 per incident. Instrument counting protocols have been implemented in an effort to reduce the rate of occurrence and at junctions between major locations through the lifecycle of an instrument in an effort to eliminate missing instrumentation. Unfortunately, instrument counts have had limited success due to human error despite requiring significant time and resources to complete. Approximately, 1 out of 8 surgical trays undergo a count discrepancy that takes an average of 20 minutes to resolve. A case-control study demonstrated that of all instances of retained foreign bodies, 88% were thought to be accounted for via manual count. This inaccuracy also incurs significant cost through lost instrumentation. Also, in some instances surgical instruments may be discarded with linens. In view of these issues, the cost, duration, and inaccuracy of manual instrument counts motivate the search for an alternative.

Oversupply, missing instrumentation, and instances of retained surgical instruments are difficult problems to solve when considering the complex hospital ecosystem. The foundation of oversupply stems from surgical preference cards and a lack of standardization. A preference card may be, for example, a listing of instruments or sets of instruments that are to be supplied to the surgeon for a particular surgery. Surgeons develop preferences for specific products or vendors early in their careers that they bring to the institution. This eliminates the possibility of standardization as each surgeon maintains a unique preference card. In theory, preference cards are meant to provide a check for correct instrument supply and to motivate reassessment of which instruments are necessary to an operation. In practice, instruments are added for a special case and are quickly forgotten, joining the majority of instruments that are supplied and cycled but are not used. Determining which instruments are important to a specific surgeon and operation is a monumental task when considering the sheer quantity of instruments in circulation. It is estimated the average 15-room OR has 3000-4000 products in multiple locations. Formerly, quality improvement projects focusing on instrument management required manual counting and observation of each instrument by personnel with plenary knowledge of names and appearances. As a result, considerable investment has been expended to quantify a problem and implement a solution.

In view of the foregoing, there is a need for improved systems for managing and tracking surgical instruments and other medical equipment.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a top diagram view of an example operating room in which a system in accordance with embodiments of the present disclosure may be implemented;

FIG. 2 is a flow diagram of an example method of medical equipment tracking and usage analysis in accordance with embodiments of the present disclosure;

FIG. 3 is a side view of an example applicator for applying electronic identification tagging tape to medical equipment in accordance with embodiments of the present disclosure;

FIG. 4 is a flow diagram of an example method for managing surgical preference cards in accordance with embodiments of the present disclosure;

FIG. 5 is a flow diagram of an example method for predicting surgical tool sharpening and maintenance in accordance with embodiments of the present disclosure;

FIG. 6A is a perspective view of an example portion of electronic identification tagging tape in accordance with embodiments of the present disclosure; and

FIG. 6B is a cross-sectional side view of a portion of a surgical instrument having electronic identification tagging tape wrapped around it in accordance with embodiments of the present disclosure.

### SUMMARY

The presently disclosed subject matter provides electronic identification tagging systems, methods, applicators, and tapes for tracking and managing medical equipment. According to an aspect, a system includes electronic identification tag readers distributed within predetermined areas of an environment. The system also includes electronic identification tags attached to respective medical equipment within the environment. Further, the system includes a computing device comprising an object use analyzer configured to receive, from the electronic identification tag readers, information indicating presence of the electronic identification tags within the predetermined areas. The object use analyzer also analyzes usage of the medical equipment within the environment based on the received information. Further, the object use analyzer manages one of medical equipment supply or usage of the medical equipment during a medical procedure based on the analyzed usage of the medical equipment.

According to another aspect, electronic identification tagging tape is disclosed. The tape includes a strip of material having an adhesive surface. Further, the tape includes electronic identification tags attached to the strip of material. The electronic identification tags are positioned apart from each other along a length of the strip of material.

According to another aspect, an applicator for electronic identification tagging tape is disclosed. The applicator includes a reel configured to hold electronic identification tagging tape having electronic identification tags positioned apart from each other and along a length of the tape. Further, the applicator includes a tape advancer configured to advance an end of the tape a predetermined length from the reel such that a single electronic identification tag is unreeled for application to medical equipment.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

In accordance with embodiments, a system is disclosed that includes multiple electronic tag readers distributed within predetermined areas of an environment, such as an OR. In an example, the electronic tag readers may be RFID readers and may be attached to or held by equipment or persons within an OR. Examples of RFID reader placement include, but are not limited to, a surgical site, an operating table, a sleeve of a medical practitioner (e.g., surgeon or surgeon's assistant), an OR doorway, a surgical instrument tray, a Mayo stand, the overhead surgical lights, and the surgical bed. The system also includes electronic identification tags. The electronic identification tags may be attached to respective medical equipment within the environment. For example, the electronic identification tags may be RFID tags attached to surgical instruments or other medical equipment. The system may also include a computing device having an object use analyzer (implemented by hardware, software, firmware, or combinations thereof). The computing device may be communicatively connected (e.g., wireless or wired connection) to the electronic identification tag readers. The object use analyzer may be configured to receive, from the electronic identification tag readers, information indicating presence and location of the electronic identification tags within the predetermined areas. Further, the object use analyzer may analyze usage of the medical equipment within the environment based on the received information. The object use analyzer may manage one of medical equipment supply or usage of the medical equipment during a medical procedure based on the analyzed usage of the medical equipment. Such a system can be used to, for example, help to solve hospital issues of oversupply, the prevalence of RSI, and missing instrumentation while minimizing the impact on surgical workflow. Example tags include, but are not limited to, image recognition algorithms, barcode technologies, RFID, engravings, and combinations thereof.

In accordance with embodiments, electronic identification tags may be RFID tags that are attached to surgical instruments or other objects such that RFID readers placed within an OR may be used to track and count the surgical instruments. RFID technology may, in this way, provide an alternative to manual counting that is cost-effective, reliable, semi-autonomous, and agnostic of surgical workflow. By attaching RFID tags to surgical instruments or other medical equipment disclosed herein, a system as disclosed herein can generate statistics on usage for reducing hospital cost and improving efficiency. Surgical instrument usage data collected by the system may be analyzed by the system to make recommendations as to which instruments should be supplied during specific surgeries for particular surgeons. For example, the system may analyze instrument usage of a particular surgeon and determine that the surgeon never uses a particular surgical instrument for a particular surgery or generally for any type of surgery. In this example, for future surgeries, the system may recommend that the surgical instrument not be supplied in future surgeries for that surgeon. In this example, the system may update the surgeon's preference card such that the surgical instrument is not included for particular surgeries or for surgeries in general based on whether the surgeon uses the surgical instrument. The implications of instrument level tracking extend farther than optimizing instrument management. By leveraging the same RFID tags and underlying technology, instrument counts and retained instrument checks can be accomplished quickly.

In accordance with embodiments, electronic identification tags can be attached to surgical instruments or other medical equipment by use of electronic identification tagging tape as disclosed herein. Electronic identification tagging tape may include a strip of material having an adhesive surface. Further, the tape may include multiple electronic identification tags attached to the strip of material. The electronic identification tags may be positioned apart from each other along a length of the strip of material. In an example, the electronic identification tags are RFID tags attached to and positioned along the length of the strip of material. The strip of material may be cut or otherwise separated between neighboring tags in order to remove an individual tag along with a portion of the strip of material it is attached to. This separated portion of the strip of material along with the individual tag may subsequently be attached to, for example, a surgical instrument such that the RFID tag may be used for tracking usage of the surgical instrument. RFID tags may be attached to all or at least some surgical instruments for use in tracking usage as described in further detail herein. Tags on a strip of material may be manufactured and transported on a reel and may be peeled off or otherwise separated and applied as tape to a surgical instrument, for example, with an RFID tag attached thereto.

As referred to herein, the term “strip” is a relatively long piece of material that may have uniform or substantially uniform width. The strip may have an adhesive surface that can be used for sealing, binding, or attaching itself and/or another object to another object. The strip of material may be flexible. In an example, the strip of material may be made of vinyl, plastic, or other suitable material. In an example, the strip of material is a 4-mil sheet of vinyl. In other examples, the strip of material may be a sheet of vinyl between about 1 and 10 mils. The strip of material and the RFID tags as applied thereto may be compatible with chemical, thermal, ultrasonic, light, and steam sterilization as surgical instrument undergo in a hospital or other medical facility. The strip of material may have a rubber adhesive attached to one side such that the strip of material may be attached to an object, such as a surgical instrument.

As referred to herein, the term “electronic identification tag” is any electronic device that can be used to identify an object associated with it. For example, an electronic identification tag may be an RFID tag, which is an electronic device that stores information, such as identification data, and uses electromagnetic fields to communicate the stored information to an RFID reader. RFID tags may be passive in that they collect energy from a nearby RFID reader's interrogating radio waves and use the energy to transmit the stored information to the interrogating RFID reader. In another example, the RFID tag may have a local power source to self-power communication of the stored information. In accordance with embodiments, the stored information may be identification of a type of surgical instrument to which the RFID tag is attached to. In accordance with embodiments, an RFID tag may be a ultra-high frequency (UHF) RFID tag having at least 3 components: an integrated circuit (IC), an antenna, and a substrate. Example characteristics of at least some of the RFID tags disclosed herein include: flexibility, adhesiveness, small size, affixable to surgical instruments, ability to be read even when attached to metallic tools, endurance to autoclaving, and low cost for manufacture. Example electronic identification tags include, but are not limited to, 3D data matrices, laser-engraved codes, bar codes, or ultrasound identification tags.

In an example, antennas for RFID tags disclosed herein may match the complex impedance of the grouped antenna and tool to the impedance of the IC. A dipole antenna, inverted F-type antenna, patch-type antenna, or meander dipole antenna may collect electromagnetic waves and transmit their power through an inductive coupling to a magnetic loop antenna soldered or epoxied to the terminals of the IC. The proximity of the magnetic loop antenna to the dipole or other type of antenna may be optimized by maximizing the impedance match of the entire antenna assembly to the IC. The shape and dimensions of the magnetic loop antenna may be selected to match the conjugate reactance of the IC. The length of the dipole antenna may be determined by even fractions of the wavelength of the mid-band frequency (e.g., 915 MHz for some antenna) and the real contribution of the input impedance of the IC. If the impedance match is not possible with direct application to a metallic instrument, a foam, ceramic, or other suitable material spacer may be used to space the antenna away from the tool.

In another example, an RFID tag may include a loop antenna for communication of the stored data. The loop may inductively or capacitively couple to the body of the surgical instrument and have dimensions that incorporate instrument-mounting effects into the impedance match of the antenna and the IC. For example, the loop antenna may be a square loop having side lengths of a range between about 3 millimeters (mm) and 60 mm. Further, for example, the loop antenna may have between 1 and 200 turns. Further, for example, the loop antenna may be a wire having a diameter between about 2 microns and 5 mm. The loop antenna may wrap around the instrument body or sit on the body of the instrument without wrapping around and contain between 1 and 200 turns.

In accordance with embodiments, flexible polyurethane (FPU) 3D filament or other FDA certified 3D filament may be used for printing a substrate with an antenna trough for filing with a stretchable silver or copper conductive paste. In this manner, the stress generated from unique thermal expansion rates may be mitigated through high elasticity of the antenna itself. The connection of the antenna to the IC may be epoxy or soldered with a high-strength bonding agent that is unlikely to fail under thermal stress. Once the trough is filled with the conductive paste and the IC epoxied in place, a top made of the same substrate material can be printed to seal the assembly in place. The RFID tag may now be functional and resistant to stretching, bending, heating, and cooling. Water resistant or water proof heat shrink tubing or tape may be used to attach the RFID tag to medical equipment, such as a surgical instrument.

In another example of RFID tag manufacture, a silicon mold may be prepared, filled with FDA certified silicon, and the antenna assembly may be submerged within it. Further, an adhesive backing may be added.

In yet another example of RFID tag manufacture, RFID tags may be built directly into an adhesive, flexible tape. This approach may include a lamination or encapsulation procedure in which the RFID antenna and circuitry is enclosed in a water-tight, electrically isolating covering and also has adhesive properties. In some cases, the film may exhibit shrinkage properties such that is can be adhered around a surgical tool by providing heat thereto.

In accordance with embodiments, an applicator is disclosed that can be used for applying electronic identification tagging tape as disclosed herein to objects. For example, the applicator may be used to apply the electronic identification tagging tape to surgical instruments. The applicator may include a reel that can hold electronic identification tagging tape as disclosed herein. Further, the applicator may include a tape advancer that can advance an end of the tape a predetermined length from the reel such that a single electronic identification tag is unreeled for application to medical equipment, such as a surgical instrument.

In accordance with embodiments, an applicator may include a computing device comprising an equipment recordation manager. The computing device may be attached to the applicator. The equipment recordation manager may receive identification of medical equipment to which one of the electronic identification tags is applied. For example, a user may input identification of the medical equipment into the equipment recordation manager. This input information may identify the medical equipment that an RFID tag (or other electronic identification tag) is be attached to by the applicator. In addition, the equipment recordation manager may associate the received identification of the medical equipment with an identifier of the RFID tag that is being attached to the medical equipment. In an example, the applicator may include an image capture device (e.g., a camera) that can capture an image of the medical equipment. In this example, the equipment recordation manager can determine the identification of the medical equipment based on the captured image. The applicator may also be connected to an existing instrument management software or database and pull instrument identification information from this data and pair it to the electronic identification tag identifier. Alternatively, for example, a user may enter user input for identifying the medical equipment.

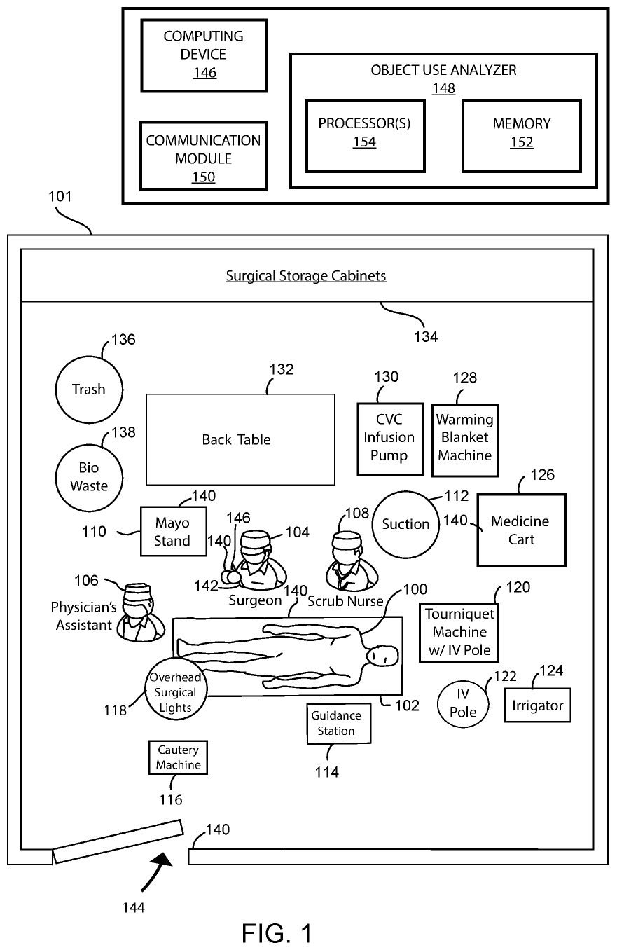

FIG. 1 illustrates a top diagram view of an example OR 101 in which a system in accordance with embodiments of the present disclosure may be implemented. It is noted that the system is described in this example as being implemented in an OR, although the system may alternatively be implemented in any other suitable environment such as a factory, dentist office, veterinary clinic, or kitchen. Further, it is noted that in this example, the placement of a patient, medical practitioners, and medical equipment are shown during surgery.

Referring to FIG. 1, a patient 100 is positioned on a surgical table 102. Further, medical practitioners, including a surgeon 104, an assistant 106, and a scrub nurse 108, are shown positioned about the patient 100 for performing the surgery. Other medical practitioners may also be present in the OR 101, but only these 3 medical practitioners are shown in this example for convenience of illustration.

Various medical equipment and other objects may be located in the OR 101 during the surgery. For example, a Mayo stand 110, a suction machine 112, a guidance station 114, a cautery machine 116, surgical lights 118, a tourniquet machine 120, an intravenous (IV) pole 122, an irrigator 124, a medicine cart 126, a warming blanket machine 128, a CVC infusion pump 130, and/or various other medical equipment may be located in the OR 101. The OR 101 may also include a back table 132, various cabinets 134, and other equipment for carrying or storing medical equipment and supplies. Further, the OR 101 may include various disposal containers such a trash bin 136 and a biologics waste bin 138.

In accordance with embodiments, various RFID readers and tags may be distributed within the OR 101. For convenience of illustration, the location of placement of RFID readers and RFID tags are indicated by reference numbers 140 and 142, respectively. In this example, RFID readers 140 are attached to the Mayo stand, the surgical table 102, a sleeve of the surgeon 104, and a doorway 144 to the OR 101. It should be understood that the location of these RFID readers 140 are only examples and should not be considered limiting as the RFID readers may be attached to other medical equipment or objects in the OR 101 or another environment. It should also be noted that one or more RFID readers may be attached to a particular object or location. For example, multiple RFID readers may be attached to the Mayo stand 140 and the surgical table 102.

An RFID tag 142 may be attached to medical equipment or other objects for tracking and management of the medical equipment and/or objects in accordance with embodiments of the present disclosure. In this example, an RFID tag 142 is attached to the non-working end of a surgical instrument 145. RFID readers 140 in the OR 101 may detect that the surgical instrument 145 is nearby to thereby track usage of the surgical instrument 145. For example, the surgical instrument 145 may be placed in a tray on the Mayo stand 110 during preparation for the surgery on the patient 100. The RFID reader 140 on the Mayo stand 110 may interrogate the RFID tag 142 attached to the surgical instrument 145 to acquire an ID of the surgical instrument 145. The ID may be acquired when the surgical instrument 145 is sufficiently close to the Mayo stand's 110 RFID reader 140. In this way, it may be determined that the surgical instrument 145 was provided for the surgery. Also, the Mayo stand's 110 RFID reader 140 may fail to interrogate the RFID reader 140 in cases in which the surgical instrument's 145 RFID tag 142 is out of range. The detection of a RFID tag 142 within communicated range is information indicative of the presence of the associated medical equipment within a predetermined area, such as on the Mayo stand 110.

It is noted that an RFID reader's field of view is dependent upon the pairing of its antennas. The range of the RFID reader is based upon its antennas and the antennas can have different fields of view. The combination of these fields of view determines where it can read RFID tags.

It is noted that this example and others throughout refer to use of RFID readers and RFID tags. However, this should not be considered limiting. When suitable, any other type of electronic identification readers and tags may be utilized.

The Mayo stand's 110 RFID reader 140 and other readers in the OR 101 may communicate acquired IDs of nearby medical equipment to a computing device 146 for analysis of the usage of medical equipment. For example, the computing device 146 may include an object use analyzer 148 configured to receive, from the RFID readers 140, information indicating presence of RFID tags 142 within areas near the respective RFID readers 140. These areas may be referred to as “predetermined areas,” because placement of the RFID readers 140 within the OR 101 is known or recognized by the object use analyzer 148. Thereby, when a RFID reader 140 detects presence of a RFID tag 142, the ID of the RFID tag 142 (which identifies the medical equipment the RFID tag 142 is attached to) is communicated to a communication module 150 of the computing device 146. In this way, the object use analyzer 148 can be informed that the medical equipment associated with the ID was at the predetermined area of the RFID reader 140 or at a distance away from the predetermined area inferred from the power of the receive signal. For example, the object use analyzer 148 can know or recognize that the surgical instrument 145 is within a predetermined area of the RFID reader 140 of the Mayo stand 110. Conversely, if the RFID tag 142 of the surgical instrument 145 is not detected by the RFID reader 140 of the Mayo stand 110, the object use analyzer 148 can know or recognize that the surgical instrument 145 is not within the predetermined area of the RFID reader 140 of the Mayo stand 110.

The RFID reader, such as the RFID readers 140 shown in FIG. 1, may stream tag read data over an IP port that can be read by a remote listening computer. The port number and TCP port number are predetermined to provide a wireless communication link between the two without physical tethering. The receiving computer may be located in the OR or outside the OR. Data can also be sent and received over Ethernet or USB.

Data about the presence of RFID tags 142 at predetermined areas of the RFID readers 140 can be used to analyze usage of medical equipment. For example, multiple different types of surgical instruments may have RFID tags 142 attached to them. These RFID tags 142 may each have IDs that uniquely identify the surgical instrument it is attached to. The object use analyzer 148 may include a database that can be used to associate an ID with a particular type of surgical instrument. Prior to beginning a surgery, the surgical instruments may be brought into the OR 101 on a tray placed onto the Mayo stand 110. An RFID reader on the tray and/or the RFID reader 140 on the Mayo stand 110 may read each RFID tag attached to the surgical instruments. The ID of each read RFID tag may be communicated to the object use analyzer 148 for determining their presence and availability for use during the surgery. In this way, each surgical instrument made available for the surgery by the surgeon 104 can be tracked and recorded in a suitable database.

Continuing the aforementioned example, the surgeon 104 may begin the surgery and begin utilizing a surgical instrument, such as a scalpel. The RFID reader 140 at the stand may continuously poll RFID tags and reported identified RFID tags to the object use analyzer 148 of the computing device 146. The object use analyzer 148 may recognize that the RFID tag of the surgical instrument is not identified, and therefore make the assumption that it has been removed from the surgical tray and being used for the surgery. The object use analyzer 148 may also track whether the surgical instrument is returned to the surgical tray. In this way, the object use analyzer 148 may track usage of surgical instruments based on whether they are detected by the RFID reader 140 attached to the Mayo stand 110.

It is noted that the object use analyzer 148 may include any suitable hardware, software, firmware, or combinations thereof for implementing the functionality described herein. For example, the object use analyzer 148 may include memory 152 and one or more processors 154 for implementing the functionality described herein. It is also noted that the functionality described herein may be implemented by the object use analyzer 148 alone, together with one or more other computing devices, or separately by an object use analyzer of one or more other computing devices.

Further, it is noted that although electronic identification tags and readers (e.g., RFID tags and readers) are described as being used to track medical equipment, it should be understood that other suitable systems and techniques may be used for tracking medical equipment, such as the presence of medical equipment within a predetermined area. For example, other tracking modalities that may be used together with the electronic identification tags and readers to acquire tracking information include, but are not limited to, visible light cameras, magnetic field detectors, and the like. Tracking information acquired by such technology may be communicated to object use analyzers as disclosed herein for use in analyzing medical equipment usage and other disclosed methods.

Referring to FIG. 1, aside from placement at the Mayo stand 110, RFID readers 140 are also shown in the figure as being placed in other locations throughout the OR 101. For example, RFID readers 140 are shown as being placed at on the operating table 102, on the surgeon's 104 sleeve, and the doorway 144. However, it is noted that the RFID readers may also be placed at other locations throughout the OR 101 for reading RFID tags attached to medical equipment to thereby track the medical equipment. Placement of RFID readers 140 throughout the OR 101 can be used for determining the presence of medical equipment in these areas to thereby deduce a use of the medical equipment, such as the described example of the use of the surgical instrument 146 if it is determined that it is no longer present at the Mayo stand 110. For example, placing an RFID reader and antenna with field of view tuned to view the doorway of the operating room can be used to know exactly what instruments enter the room. Knowing the objects that entered the room can be used for cost recording, as CPT codes can be automatically called.

Some antenna characteristics of RFID readers that can be important to the uses disclosed herein include frequency, gain, polarization, and form factor. For applications disclosed herein, an ultra-high frequency, high gain, circularly polarized, mat antenna may be used. There are three classes of RFID frequencies: low frequency (LF), high frequency (HF), and UHF. UHF can provide the longest read range among these three, and may be utilized for the applications and examples disclosed herein. Understanding that small sized RFID tags may need to be used to fit some medical equipment such as surgical instruments, UHF may be used to provide the longest read range of the three. A mixture of high and low gain reader antennas may be utilized as they allow for either longer communication range and limited span of the signal or vice versa. Choosing one or the other may be important for reading specific field of views that are contingent on desired outcomes.

There exist two classes of polarized antennas: circular and linear. Linear polarization can allow for longer read ranges, but tags need to be aligned to the signal propagation. Circularly-polarized antennas may be used in examples disclosed herein as surgical tool orientation is random in an OR.

The form factor of most antennas may be a mat, as they can be laid underneath a sterile field, patient, instrument tables, central sterilization and processing tables, and require little space. Their positioning and power tuning allow for a limited field of view encompassing only instruments that enter their radiation field. This characteristic may be desirable because instruments can be read by an antenna focused on the surgical site, whereas instruments that are on back tables cannot be read. For tool counting within trays or across the larger area of a table away from the surgical site, an unfocused antenna may be desirable. This type of setup allows for detection of the device within the field of interest.

When an instrument is detected within a field of interest via an RFID tag read, it may be referred to as an “instrument read”. Instrument reads that are obtained by the antenna focused on the surgical site (e.g., surgical table 102) may be marked as “used instruments” and others being read on instrument tables are not. Some usage statistics may also be inferred from the lack of instrument reads in a particular field.

In accordance with embodiments, mat antennas may be placed under surgical drapes, on a Mayo stand, on instrument back tables, or anywhere else relevant within the OR or within the workflow of sterilization and transportation of medical equipment (e.g., surgical instruments) for real-time or near real-time medical instrument census and counts in those areas. Placement in doorways (e.g., doorway 144) can provide information on the medical equipment contained in a room. Central sterilization and processing (CSP) may implement antennas for censusing trays at the point of entry and exit to ensure their contents are correct or as expected. The UHF RFID reader may contain multiple antenna ports for communication with multiple antennae at unique or overlapping areas of interest (e.g., the surgical site, Mayo stand, and back tables). The reader may connect to software or other enabling technology that controls power to each antenna and other pertinent RFID settings (such as Gen2 air interface protocol settings), tunable for precise read rate and range. Suitable communication systems, such as a computer, may subsequently broadcast usage data of an Internet protocol (IP) port to be read by a computing device, such as computing device 146. The data may be saved locally, saved to a cloud-based database, or otherwise suitably logged. The data may be manipulated as needed to derive statistics prior to logging or being stored.

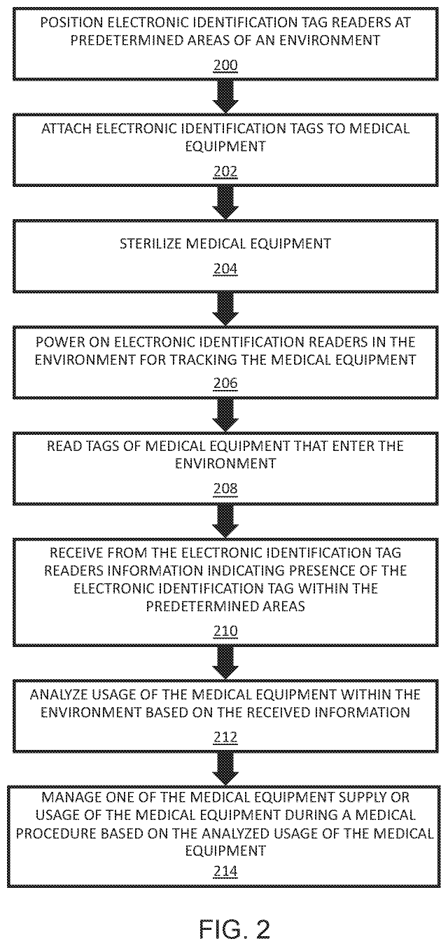

In accordance with embodiments, FIG. 2 illustrates a flow diagram of an example method of medical equipment tracking and usage analysis in accordance with embodiments of the present disclosure. This method is described as being implemented by the system and within the OR 101 shown in FIG. 1. However, it should be noted that the method may alternatively be implemented by another suitable system in a different OR or environment.

Referring to FIG. 2, the method includes positioning 200 one or more electronic identification readers at predetermined areas of an environment. For example, RFID readers 140 may be distributed within predetermined areas of the OR 101 as shown in FIG. 1. The RFID readers may be configured to read RFID tags 142 located within their respective predetermined areas.

The method of FIG. 2 includes attaching 202 electronic identification tags to medical equipment. Continuing the aforementioned example, the RFID tag 142 may be suitably attached to the surgical instrument 146. In examples disclosed herein RFID tags 142 may be attached to medical equipment, such as surgical instruments, by use of an applicator for attaching electronic identification tags. Example applicators for attaching electronic identification tags are described in more detail herein. The electronic identification tag may include an electronic product code (EPC) that, once read and its data communicated to the computing device 146, can be used by the object use analyzer 148 to link the name and type of medical equipment (e.g., surgical instrument) in a database either in central sterilization and processing (CSP) or at an earlier point in the medical equipment acquisition pipeline. Memory of 152 of the object use analyzer 148 may identify and correlate each RFID tag to particular medical equipment and an instance of it. In an example, the object use analyzer 148 may store an image of the medical equipment and control a display to display the image as well as other information to ensure the medical equipment is correctly identified. Kits or other storage units containing tagged medical equipment, such as surgical instruments, may be tagged with an RFID tag. An instrument can be packaged and scanned in its storage unit (e.g., tray) by an antenna to ensure the contents of the storage unit are correct. The object use analyzer 148 may function to receive scanning data and to indicate expected contents of the storage unit.

The method of FIG. 2 includes sterilizing 204 the medical equipment. Continuing the aforementioned example, RFID-tagged surgical instruments may be sterilized. Sterilization may be monitored by RFID antennas positioned on either side of an autoclave to account for each surgical instrument being sterilized. The gathering of this information can help to ensure that each surgical instrument completes the sterilization process. The memory 152 may store an inventory of the tags of surgical instruments that have gone through the sterilization process and have been read for comparison to tags of surgical instruments in a tray. The object use analyzer 148 may compare the list of the sterilized instruments to the inventor of the tray to determine whether any in the tray have not been sterilized. Those that have not been sterilized may be reported to medical practitioners, so the non-sterilized instrument are not used in surgery. In an example of the sterilization process, the time of reads on either side of a sterilization autoclave may be used to determine a go/no-go gauge on delivery. An RFID-tagged tray containing RFID-tagged instruments may subsequently be scanned as it leaves CSP to track the tray.

The method of FIG. 2 includes powering on 206 electronic identification readers in the environment for tracking the medical equipment. Continuing the aforementioned example, subsequent to detection of the RFID tags and determining that the surgical instruments and/or other medical equipment have entered the OR 101, the object use analyzer 148 may use the communication module 150 to power on RFID antennas in the OR 101. The RFID readers 140 may subsequently begin monitoring their field of views and logging data. In an example, the RFID readers 140 may be power via a wall electrical outlet or other suitable power source. Example tag read data may include, but is not limited to, time of read, EPC, and strength of read. Such tag read data may be parsed and stored in a database within the memory 152. Further, the object use analyzer 148 may associate with the tag read data a type of surgery in the OR 101, the OR number, identity of the surgeon 104 performing the surgery, the surgical team performing the surgery, the like, and/or any other suitable data.

The method of FIG. 2 includes reading 206 tags of medical equipment that enter the environment. Continuing the aforementioned example, the RFID reader 140 at doorway 144 may detect one or more RFID tags. The RFID reader 140 at the doorway may communicate to the computing device 146 the IDs of the RFID tags. The object use analyzer 148 may update the database at memory 152 to indicate that the surgical instruments with the IDs have entered the OR 101.

The method of FIG. 2 includes receiving 210, from the electronic identification tag readers, information indicating presence of the electronic identification tags within the predetermined areas. Continuing the aforementioned example, the RFID readers 140 within the OR 101 shown in FIG. 1 may read data from the RFID tags 142 in the RFID readers' 140 respective areas. The read tag data may be communicated to the communication module 150 of the computing device 146. The object use analyzer 148 may thereby receive the read tag data, which can be indicative of the presence of RFID tags within the areas of the RFID readers. The received tag data may be stored in the memory 152.

The method of FIG. 2 includes analyzing 212 usage of the medical equipment within the environment based on the received information. Further, the method of FIG. 2 includes managing 214 one of medical equipment supply or usage of the medical equipment during a medical procedure based on the analyzed usage of the medical equipment. Continuing the aforementioned example, the object use analyzer 148, either during or post-surgery, may call data from the database in memory 152 and prepare and present overarching statistics on whether one or more surgical tools and/or other medical equipment was present in the surgical site, the amount of time the medical surgical instrument(s) and/or other medical equipment was present, and other actionable analytics that may be unique to each operation and surgeon. Such data may be gathered from multiple surgeries to generate analytics that may be of more significance than a single surgery. In an example of managing medical equipment supply and usage of the medical equipment, surgical preference cards may be generated and/or changed by statistical significance and client-decided thresholds for cutoff percentages. The object use analyzer 148 may be configured to determine a utilization metric for one or more of the medical equipment (e.g., surgical instrument). The utilization metric can be generated over a variety of operations with any number of different surgeons. The metric can include the number of times a specific instrument was used, a risk metric correlated to how much the surgery would be impacted if a specific instrument was not supplied, and a cost metric that reflects the cost of supplying a specific instrument.

In an example, surgical instruments used 0-15% of the time for particular surgeries and/or by particular surgeons may not be used in future surgical procedures. In this example, such surgical instruments may not be included on preference cards for these surgeries and/or surgeons. Further, in these cases, the object use analyzer 148 may update the preference cards for these surgeries and/or surgeons to remove surgical instruments in which it is determined they are only used 0-15% of the time.

In other examples in which it is determined surgical instruments are used 16-30% of the time for particular surgeries and/or by particular surgeons, such surgical instruments may be stored on hand in peel packs to reduce the number of necessary sterilization cycles. In these cases, the object use analyzer 148 may update the preference cards for these surgeries and/or surgeons to indicate that such surgical instruments are to be kept on hand in peel packs.

In other examples in which it is determined surgical instruments are used 31-100% of the time for particular surgeries and/or by particular surgeons, such surgical instruments may be stored as usual and as such indicated on preference cards. In these cases, the object use analyzer 148 may update the preference cards for these surgeries and/or surgeons to indicate that that the surgical instruments are to be made available for surgeries.

It is noted that the aforementioned example cutoff percentages may be decided by hospital management and may vary depending on the hospital. A user of the computing device 146 may use a user interface to enter the cutoff percentages. Once stable cutoff values are defined, kits can be organized by percent usage to a specific surgery and standardized between surgeons.

In accordance with embodiments, medical practitioners may use a computing device to census areas within an environment. For example, referring to FIG. 1, the physician's assistant 106 may use the computing device 146 to census areas of the OR 101, such as the back table 132 or the Mayo stand 140. In this example, the physician's assistant 106 may interact with the computing device 146 via a user interface (e.g., keyboard and mouse) to command the object use analyzer 148 to census RFID readers 140. The object use analyzer 148 may receive input that identifies one or more surgical instruments or other medical equipment. In response, the object use analyzer 148 may poll RFID readers 140 to receive indication of presence of RFID tags. The object use analyzer 148 may determine a location of the user-identified surgical instrument(s) based on the returned polling data. Subsequently, the object use analyzer 148 may control a display or other user interface to indicate the location of the user-identified surgical instrument(s). Further, the computing device 146 may present information on OR monitors and provide immediate verification or waning of instrument presence. Further, the system may be used in training module programs for new practitioners to display the name and picture of a tool as it is picked up for improving the accuracy of tool delivery.

After a surgery for example, various data may be input into the computing device 146 about the medical equipment used during the operation. For example, the computing device 146 user may input data indicating that a particular instrument is broken or dull, and this information may be associated with the ID for the instrument. Further, instruments may be placed back in their respective trays. Each tray may be scanned to verify that it contains the correct instruments before being returned to CSP for sterilization. In addition, an RFID reader may be used to ensure no tool is left behind in the surgical field (i.e., in the patient). In CSP, dull and broken instruments may be replaced with newly tagged instruments. As instruments travel through this cycle, metrics on the number of cycles a tool passes through may be recorded. When instruments are marked as dull or broken, this can inform the object use analyzer 148 that supplies recommendations for scheduled maintenance or replacement on other similar instruments. It is also noted that optimal manufacturing and purchasing scheduling can be recommended for future purchasing based on the longevity of instruments or other medical equipment.

In accordance with embodiments, the object use analyzer 148 may be configured to determine an operational procedure associated with use of surgical instruments, and subsequently predict surgical instruments needed for a subsequent operational procedure based on the determined operational procedure and the usage of the surgical instruments. For example, the object use analyzer 148 may receive information about one or more surgical procedures and usage of surgical instruments during the procedure(s). The object use analyzer 148 may predict whether the same or similar procedures need the surgical instruments based on the usage of the surgical instruments during previous procedures.

In accordance with embodiments, the object use analyzer 148 is configured to store information that indicates a standard order and timing of use of the medical equipment during a medical procedure. For example, the object use analyzer 148 may store information about an ideal order of use of surgical instruments and timing of use of the surgical instruments during a surgical procedure. Further, the object use analyzer 148 may determine whether the medical equipment is being used in accordance with the stored order and timing. Continuing the example, the object use analyzer 148 may determine whether the surgical instruments are being used in accordance with the stored order and timing. The object use analyzer 148 may subsequently present, to a medical practitioner such as the surgeon 104, information that indicates whether the surgical instruments are being used in accordance with the stored order and timing. The use analyzer can also present to the nursing staff which subsequent instruments it anticipates will be needed in the future. This can be used to increase the efficiency of the surgical team. This may be presented by the object use analyzer 148, for example, by displaying the standard use progression to the nursing team with an indicator of the current stage of the surgery. This information may also be displayed as a picture and name of the instrument next anticipated to be needed.

In accordance with embodiments, the object use analyzer 148 may determine, based on information received from RFID tags attached to medical equipment (e.g., surgical instruments), signatures of use of the medical equipment by a plurality of medical practitioners during associated medical procedures. Further, the object use analyzer 148 may determine outcome metrics for the associated medical procedures. In addition, the object use analyzer 148 may analyze the outcome metrics and signatures of use to determine preferred techniques for the medical procedures. As an example, the object use analyzer 148 may determine a timing and/or ordering of the use of surgical instrument during operations based on received RFID tag data about the surgical instruments. The timing and/or ordering may be considered a “signature” of use of the surgical instruments by one or more surgeons during a surgery. In this example, the object use analyzer 148 may receive information about outcome metrics for the operations. Subsequently, the object use analyzer 148 may analyze the outcome metric for the operations to determine preferred techniques for future operations or other medical procedures.

In accordance with embodiments, the object use analyzer 148 may be configured to determine, based on information received from RFID tags attached to surgical instruments (or other medical equipment), sterilization practices for the surgical instruments. The object use analyzer 148 may also receive information about outcome metrics for medical procedures that have used these sterilized surgical instruments. Further, the object use analyzer 148 may analyze the sterilization practices and the outcome metrics to determined preferred techniques for sterilizing the surgical instruments.

In accordance with embodiments, the object use analyzer 148 may determine, based on information received from RFID tags attached to surgical instruments, placement of the surgical instruments in one or more surgical trays during one or more surgeries. Further, the object use analyzer 148 may determine outcome metrics for the surgeries. The object use analyzer 148 may also analyze the determined placement of the surgical instruments and the outcome metrics to determine preferred surgical instrument placement on the surgical trays.

In accordance with embodiments, the object use analyzer 148 may determine, based on information received from RFID tags attached to surgical instruments, a time for notification about placement of one or more medical equipment. Further, the object use analyzer 148 may present the notification to a medical practitioner. For example, the object use analyzer 148 may determine when to use a surgical instrument during a surgery. The object use analyzer 148 may have information about order and timing of use of surgical instruments. In response to determining that it is time to use a surgical instrument, the object use analyzer 148 may control a user interface (e.g., display) to present the notification to a medical practitioner (e.g., the surgeon 104 or physician's assistant 106). The object use analyzer 148 may also present a notification that indicates that a surgical instrument has been misplaced during the surgery.

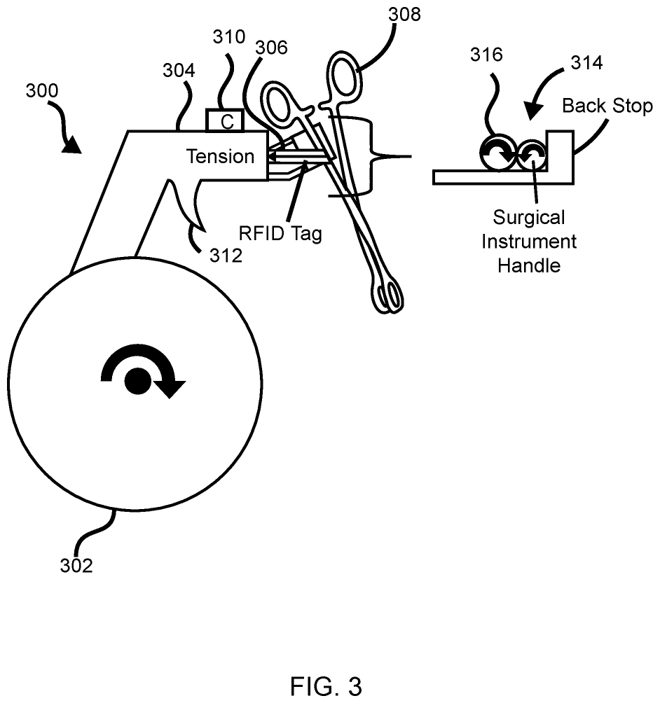

In accordance with embodiments, FIG. 3 illustrates a side view of an example applicator 300 for applying electronic identification tagging tape to medical equipment. The applicator 300 may be used to rapidly attach RFID tags to surgical instruments. Referring to FIG. 3, the applicator 300 may include a reel 302 configured to hold electronic identification tagging tape. The tape may include electronic identification tags that are positioned apart from each other and along a length of the tape. Further, the applicator 300 may include a tape advancer 302 configured to advance an end 306 of the tape a predetermined length from the reel 302 such that a single RFID tag is unreeled for application to a surgical instrument 308.

In accordance with embodiments, an object use analyzer, such as the object use analyzer 148 shown in FIG. 1, may include an equipment recordation manager (e.g., hardware, software, firmware, or combinations thereof) configured to receive identification of medical equipment (e.g., surgical instrument 308) to which one of the electronic identification tags in the reel 302 is applied. Further, the equipment recordation manager may associate identification of the medical equipment with an identifier of the electronic identification tag. For example, the applicator 300 may attach an RFID tag to the surgical instrument 308. In this example, a camera 310 or other type of image capture device may be a component of the applicator 300 and used to capture an image of the surgical instrument 308. The equipment recordation manager may determine identification of the surgical instrument 308 based on the captured image. Subsequently, the identification of the surgical instrument 308 and the RFID tag ID may be communicated to the object use analyzer 148 for later associating the surgical instrument 308 with the read RFID tag. Alternatively, for example, rather than capturing an image to identify the surgical instrument, a user may enter user input into the equipment recordation manager for identification of the surgical instrument 308.

In accordance with embodiments, the applicator 300 may include a user trigger 312 operatively connected with the tape advancer 304 and configured to effect, by the tape advancer 304, advancement of the end 306 of the tape a predetermined length. The predetermined length may be such that tape having only one RFID tag extends for cutting or other type of detachment from the reel 302. The applicator 300 may include a cutter configured to cut the tape at a space between neighboring RFID tags. The tape advancer 304 may advance the tape such that the space is positioned for cutting by the cutter.

In accordance with embodiments, the applicator 300 may include a tension mechanism 314 configured to pull the end 306 of the tape at a predetermined force such that tension on the tape is maintained while the tape is applied to the surgical instrument 308. This can ensure a tight wrapping and secure attachment of the tape to the surgical instrument 308. For example, the reel 302 may be connected to a motor such that it can turn the reel to resist pulling of the end 306 from the reel. In addition, a motorized wheel 316 of the tension mechanism can pull the end 306 to oppose the pull by the motor attached to reel 302. The two motorized systems may work together maintain tension on the tape while the tape is being applied to a surgical instrument. Friction between wheel 316 and tension mechanism 314 force the surgical instrument handle to turn by pressing wheel 316 against tension mechanism 314 against the back stop. As wheel 316 turns, tension mechanism 314 must also turn in the opposite direction. This turn rate is slightly slower than the rate of tape advancement, achieving a constant tension on the tape as the instrument turns and wraps the tape around itself.

In an example, the user trigger 312 can be pulled for advancing tape a length from the reel 302 such that an RFID tag at the end can be applied to a surgical instrument. Depending on the shape of the surgical tool, the applicator 300 may either use surgical tape or waterproof heat shrink to adhere the RFID tag to the surgical tool.

In accordance with embodiments, an object use analyzer, such as the object use analyzer 148 shown in FIG. 1, may process logged tool usage data and surgery information. The surgery information may include, but is not limited to, the names of operating surgeons, the date and duration of the surgery, the type of surgery, etc. Tool usage statistics may be obtained per surgeon per procedure. This list may be generated and sent to an appropriate party as an updated surgical preference card for the next surgery of the same or similar type. New surgeons or residents may be provided preference cards from the same or similar surgery types accomplished by senior surgeons as recommendations to their own preference. Preference cards may be updated at discrete intervals or continuously, between each surgery, with each iteration improving the preference card and correlated tool selection. CSP, surgeons, residents, management, and any other party of interest may be granted access to preference cards to improve processes.

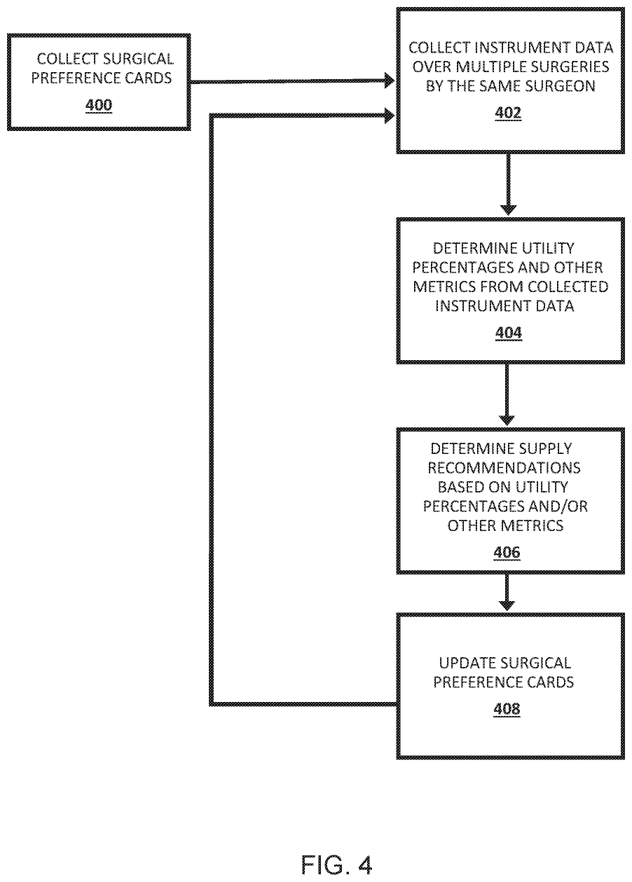

FIG. 4 illustrates a flow diagram of an example method for managing surgical preference cards in accordance with embodiments of the present disclosure. The object use analyzer 148 shown in FIG. 1 may implement the method, but it should be understood that the method may be implemented by any suitable computing device.

Referring to FIG. 4, the method includes collecting 400 surgical preference cards. For example, preference cards from one or more surgeons may be collected. The preference cards may be stored in memory 152 shown in FIG. 1. Preference cards may include instrument trays, being both general and specific to that surgeon or service line. Trays can contain a predetermined set of instruments. Preference cards may also include individual instruments.

The method of FIG. 4 includes collecting 402 instrument data over multiple similar surgeries by the same surgeon. Continuing the aforementioned example, RFID readers 140 may collect data from RFID tags 142 as disclosed herein. The collected RFID tag data may be communicated to the object use analyzer 148 and stored in memory 152.

The method of FIG. 4 includes determining 404 utility percentages and other metrics from collected instrument data. Continuing the aforementioned example, utility percentages may be determined based on the collected RFID tag data as disclosed herein. Another metric that may be factor in to utility percentage may be the cost of supplying the instrument or a metric for the degree of danger that could occur if it was not supplied.

The method of FIG. 4 includes determining 406 supply recommendations based on utility percentages and/or other metrics. Continuing the aforementioned example, items with low percent utility are not supplied in preference cards, and items with moderate percent utility instruments are supplied in peel packs. For example, instruments used between 0 and 5% of the time could be removed from a tray, while instruments used between 5 and 15% of the time could be supplied in peel packs or specialty trays. Instruments with utilities between 15 and 100% could remain in the trays and continue to be supplied.

The method of FIG. 4 includes updating 408 surgical preference card(s). Continuing the aforementioned example, tray contents can be optimized based on utility percentages over individual surgeons or entire service lines and departments. For example, if surgeon one uses 30% of the general tray, and surgeon two uses the same 30% and an additional 5% of the same tray, the general tray may be reduced to only that common 30% utility with surgeon two being supplied a separate specialized tray containing the additional 5%. Reorganization can be based on utility metrics, cost, a safety metric, the like, and combinations thereof. Preference cards may be constructed across surgeons or even service lines where overlapping usages are found to create “common trays” that reduce overall tray assembly and increase efficiency through standardization.

In accordance with embodiments, the present disclosure may be used for surgical tray organization. This may be implemented by the object use analyzer 148 shown in FIG. 1 may implement the method, but it should be understood that the method may be implemented by any suitable computing device. In an example, an initial guess may be provided for which surgical instruments are to be place in a tray. Subsequently, trays with these surgical instruments may be provided to multiple surgical operations. A sensor (e.g., RFID reader) may be used to record which instruments are used during each operation. Subsequently, a co-utilization metric may be calculated between each tool and every other tool as a function of how often each pair of tools are used during the same operation. A recommendation may be generated for which tools belong together based on the co-utilization metric that stems from common usage from unique surgeons. Subsequently, the surgical tray organization may be reorganized based on the results.

In accordance with embodiments, a method for optimizing surgical instrument tray organization may include providing an initial guess at which instruments should be placed in a tray. The method may also include supplying the tray to multiple surgical operations. Further, the method may include utilizing a sensor to record which instruments are used during each operation. The method may also include calculating a cost of sterile processing. Further, the method may include generating a recommendation for which tools belong in trays and which belong in separate sterile packaging based on the cost metric. The surgical tray may then be modified based on the recommendation.

In accordance with embodiments, systems and methods are provided for predicting surgical tool sharpening and maintenance. The methods may be implemented, for example, by the object use analyzer 148 shown in FIG. 1, or by any suitable computing device. By learning from past cycle durations and the sharpening and maintenance record of each specific type of tool, the object use analyzer 148 may provide a schedule for future sharpening and maintenance. When the appropriate time has come, CSP may receive a message prompt to sharpen a specific tagged tool or order a new tool.

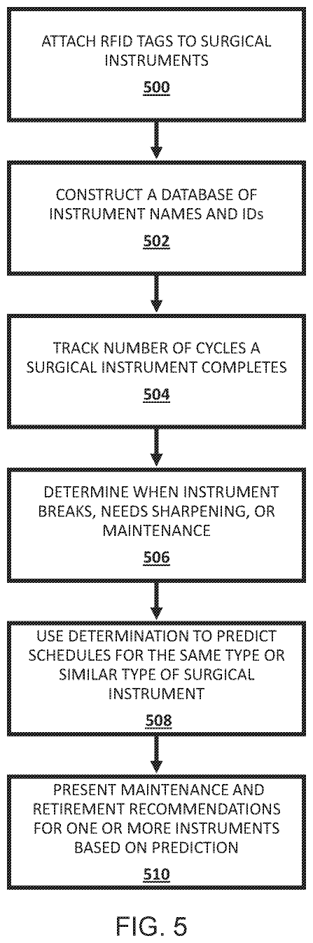

FIG. 5 illustrates a flow diagram of an example method for predicting surgical tool sharpening and maintenance in accordance with embodiments of the present disclosure. Referring to FIG. 5, the method includes attaching 500 RFID tags to surgical instruments. The method also includes constructing 502 a database of instrument names and identifiers. The database may be stored in memory 152.

The method of FIG. 5 also includes tracking 504 the number of cycles a surgical instrument completed. An example cycle can include, but is not limited to, sterilization of the instrument, being stored until a case is booked, being supplied in that case, and subsequently returned to sterilization.

The method of FIG. 5 also includes determining 506 when an instrument breaks, needs sharpening, or other maintenance. In an example, technicians in central sterilization and processing or nurses in the operating room record when an instrument needs to be replaced or maintained. This information can be collected and used to predict maintenance schedules based on the number of cycles and instrument completes.

The method of FIG. 5 also includes using 508 the determination to predict schedules for the same type or similar type of surgical instrument. As many instruments are made of similar material and have make of congruent manufacturer, recommendation s can be made across families of instruments for maintenance scheduling and be used to recommend instrument types for future purchases based on longevity.

The method of FIG. 5 also includes presenting 510 maintenance and retirement recommendations for one or more instruments based on the prediction. If the predicted lifecycle of the instrument is close to expired, the instrument be removed from supply. As a result of the method, for example, dull or broken instruments can be eliminated to thereby reduce adverse effects from unforeseen failure during operations. Instruments of a long lifetime and lasting performance can be identified and future purchasing can be targeted to corresponding manufacturers and products.

In accordance with embodiments, systems and methods are provided for an instrument training module for healthcare practitioners. The methods may be implemented by a suitable computing device, such as the computing device 146 with the equipment use manager 148. In an example, RFID tagged tools or other medical equipment can enable a learning module to display the name and an image of each tool as it enters or exits a RFID tag antenna's field of view. As one leaves or a nurse picks it up, a display of its identity can be displayed on a computer screen. This can help new nurses supply surgeons with the correct tools. If nurses know the name of the tool, but do not know which tool it describes, they can query the system to display an image of the tool as well as locate the tool with a light that illuminates the area where the tag tool is present (e.g., located via RFID signal strength). A check that all of the correct tools are present can be completed by cross referencing the surgical preference card with the census of the instrument tables. Nurses can be provided with a warning if there is any discrepancy between what was requested and what is provided.

In accordance with embodiments, the equipment use manager 148 may keep track of individual instrument usage in a database within memory 152. The individual instruments may be indicated in the database as being used on people with various instrument-transmittable conditions, such as Creutzfeldt-Jakob disease and HIV. Central sterilization and processing department technicians or nurses in the operating room may record when an instrument is used on patients with infectious diseases. These instruments can be removed from circulation and marked for further processing requirement.

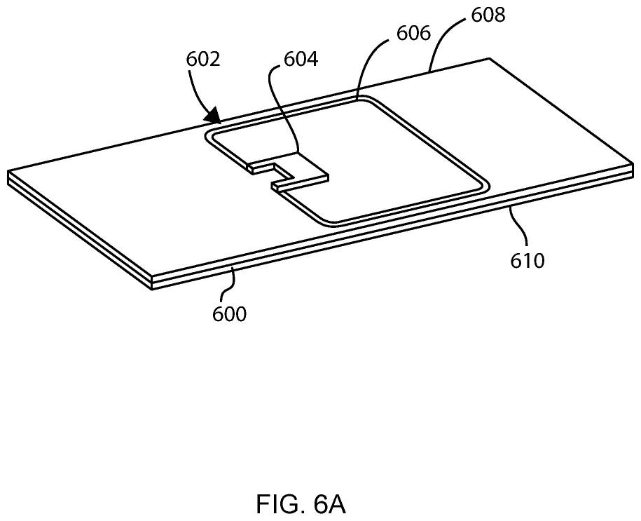

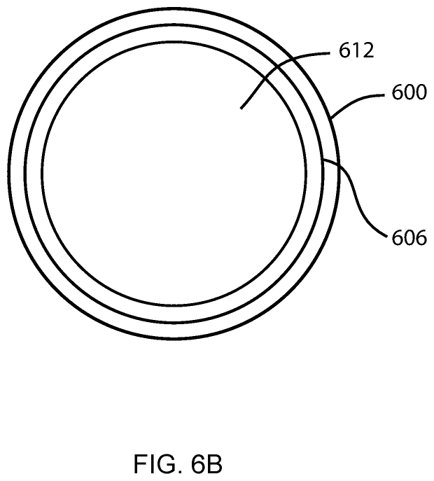

FIG. 6A illustrates a perspective view of an example portion of electronic identification tagging tape 600 in accordance with embodiments of the present disclosure. Referring to FIG. 6A, the tape 600 includes an RFID tag, generally designated 602, including an integrated circuit 604 and a copper loop antenna 606 in accordance with description provided herein. It is noted that additional RFID tags may be similarly attached and positioned along the length of the tape 600; however, for convenience of illustration only one RFID tag 602 is shown. The tape 600 may include a strip of material 608 having an adhesive surface 610. The strip of material 608, in this example, may be a vinyl 4-mil sheet. The adhesive of the surface 610 may be a rubber adhesive.

FIG. 6B illustrates a cross-sectional side view of a portion of a surgical instrument 612 having electronic identification tagging tape 600 wrapped around it in accordance with embodiments of the present disclosure. The surgical instrument 612 may be scissors, and the shown portion may be made of stainless steel. In this figure, the antenna 606 of one of the RFID tags of the tape 600 wraps around the shown portion of the surgical instrument.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

1. A system comprising:
at least one electronic identification tag reader located within at least one predetermined area of an environment;
an electronic identification tag attached to an object within the environment; and
a computing device comprising an object use analyzer configured to:
receive, from the electronic identification tag readers, information indicating presence and location of the electronic identification tag within the at least one predetermined area; and
analyze usage of the object within the environment based on the received information.

2. The system of claim 1, wherein the electronic identification tag is a radio frequency identification (RFID) tag.

3. The system of claim 1, wherein the object is one of medical equipment or a surgical instrument.

4. The system of claim 1, wherein the environment is an operating room.

5. The system of claim 1, wherein the object use analyzer determines a preference card based on the analysis of the usage of the object.

6. The system of claim 1, wherein the object use analyzer is configured to determine a utilization metric for the object.

7. The system of claim 6, wherein the utilization metric includes the number of times an object was used in a particular operation by a specific surgeon, a risk metric associated with supply of the object, and a cost metric for supply of the object.

8. The system of claim 1, wherein the environment is an operating room environment,
wherein the object is a surgical instrument, and
wherein the object use analyzer is configured to:
determine an operational procedure or medical practitioner associated with use of the surgical instrument in the operating room; and
predict surgical instruments needed for a subsequent operational procedure based on the determined operational procedure or medical practitioner and the usage of the surgical instrument.

9. The system of claim 1, wherein the at least one predetermined area includes one of a surgical site, a surgical instrument tray, an operating room doorway, a sleeve of a medical practitioner, a Mayo stand, or a surgical back table.

10. The system of claim 1, wherein the object use analyzer is configured to:
store information that indicates an order and timing of use of the object during a medical procedure;
determine whether the object is being used in accordance with the order and timing; and
present, to a medical practitioner, information that indicates whether the object is being used in accordance with the order and timing.

11. The system of claim 10, wherein the object use analyzer is configured to present, during a surgery of the same type and the same surgeon, the information to indicate progression of object usage.

12-29. (canceled)

30. Electronic identification tagging tape comprising:
a strip of material having an adhesive surface; and
a plurality of electronic identification tags attached to the strip of material and having an antenna, wherein the electronic identification tags are positioned apart from each other along a length of the strip of material, and wherein the antenna has a predetermined impedance for matching an impedance of a surgical instrument.

31-45. (canceled)

46. An applicator for electronic identification tagging tape, the applicator comprising:
a reel configured to hold electronic identification tagging tape having electronic identification tags positioned apart from each other and along a length of the tape; and
a tape advancer configured to advance an end of the tape a predetermined length from the reel such that a single electronic identification tag is unreeled for application to medical equipment.

47. The applicator of claim 46, further comprising a computing device comprising an equipment recordation manager configured to:
receive identification of medical equipment to which one of the electronic identification tags is applied; and
associate identification of the medical equipment with an identifier of the one of the electronic identification tags.

48. The applicator of claim 47, wherein the computing device further comprises an image capture device for capturing an image of the medical equipment, and
wherein the equipment recordation manager is configured to determine the identification of the medical equipment based on the captured image.

49. The applicator of claim 47, wherein the computing device further comprises a user input for receipt of identification of the medical equipment.

50. The applicator of claim 47, wherein the computing device further comprises a communication link between the computing device and an existing instrument database and pulls instrument identifiers from the database and pairs them with the tag identifier in a new database.

51. The applicator of claim 46, further comprising a user trigger operatively connected with the tape advancer and configured to effect, by the tape advancer, advancement of the end of the tape the predetermined length.

52. The applicator of claim 45, further comprising a cutter configured to cut the tape at a space between neighboring electronic identification tags.

53. The applicator of claim 52, wherein the tape advancer is configured to advance the tape such that the space is positioned for cutting by the cutter.

55-64. (canceled)
